(19) United States
(12) Patent Application Publication
(10) Pub. No.: US 2017/0095578 A1
(43) Pub. Date: Apr. 6, 2017

(54) CT SCANS USING GADOLINIUM-BASED CONTRAST AGENT

(71) Applicant: UNIVERSITY OF MASSACHUSETTS MEDICAL SCHOOL, BOSTON, MA (US)

(72) Inventors: Adib Raphael KARAM, Shrewsbury, MA (US); Andrew KARELLAS, Grafton, MA (US)

(21) Appl. No.: 15/382,511

(22) Filed: Dec. 16, 2016

Related U.S. Application Data
(63) Continuation of application No. PCT/US15/35730, filed on Jun. 15, 2015.
(60) Provisional application No. 62/013,351, filed on Jun. 17, 2014.

(51) Int. Cl. A61K 49/04; A61B 6/03; A61B 6/00

(52) U.S. Cl. CPC A61K 49/04; A61B 6/461; A61B 6/481; A61B 6/032; A61B 6/5205; A61B 6/4085

(57) ABSTRACT

A method of diagnosing a condition of a living subject that uses gadoxeate disodium as a contrast agent for making images such as CT scans of the biliary tree and related anatomical structures. The method uses x-ray radiation generated with excitation voltages in the range of 70 KV to 140 KV. The x-ray radiation is preferably filtered to suppress or practically remove x-rays having energy lower than 50.2 KeV.

### CROSS-REFERENCE TO RELATED APPLICATIONS

This application is a continuation of co-pending International Patent Application No. PCT/US15/35730, filed Jun. 15, 2015 and claims the priority and benefit thereof, which application in turn claims priority to and the benefit of then co-pending U.S. provisional patent application Ser. No. 62/013,351, filed Jun. 17, 2014, each of which applications is incorporated herein by reference in its entirety.

### FIELD OF THE INVENTION

The invention relates to x-ray methods in general and particularly to computed tomography (CT) and tomosynthesis scans.

### BACKGROUND OF THE INVENTION

Acute cholecystitis (inflammation of the gallbladder) is a very common condition, caused by blockage of the cystic duct. In 90% of the cases acute cholecystitis is caused by gallstones in the gallbladder obstructing the cystic duct, which can cause pain and discomfort. Prompt diagnosis after the onset of symptoms is very important in order to avoid complications. Ultrasonography (US) is the most commonly used imaging modality to diagnose acute cholecystitis. The reported sensitivity and specificity of US have a wide range of 48%-100% and 64%-100%, respectively. Specific limitations include poor image quality in obese patients, inability to detect sonographic Murphy's sign (pain resulting from direct compression over the gallbladder using the ultrasound transducer) in obtunded or medicated patients, nonspecificity of gallbladder wall thickening, and lack of functional information regarding cystic duct patency. Cholescintigraphy, a nuclear medicine procedure where a radioactive isotope (radiopharmaceutical) is injected intravenously has an accuracy of 92% for acute cholecystitis and has the advantage of providing functional information regarding the patency of the cystic duct and is considered the gold standard imaging modality used when the other imaging studies are inconclusive.

Therefore, there is a need for an accurate second line imaging modality to make this diagnosis in equivocal cases.

### SUMMARY OF THE INVENTION

According to one aspect, the invention features a method of diagnosing a medical condition of a living subject. The method comprises the steps of: injecting into a blood vessel of a living subject suspected to be experiencing acute cholecystitis an effective dose of a material comprising gadoxetate disodium; selecting suitable x-ray exposure parameters including at least one of beam collimation, x-ray tube current, x-ray tube voltage exposure time, and x-ray beam filtration; generating x-ray radiation; filtering the x-ray radiation to produce filtered x-ray radiation substantially lacking in radiation corresponding to an energy below 50.2 KeV; subjecting the subject to the filtered x-ray radiation; generating an image of the biliary tree and related anatomy of the living subject; determining whether a blockage of the cystic duct is present from the image; and making a diagnosis of the condition of the living subject based on the determination.

In one embodiment, the method further comprises the step of recording the image, transmitting the image to a data handling system, or to displaying the image to a user.

In another embodiment, the step of generating x-ray radiation comprises generating x-rays produced by a source operating at a voltage in the range of 70 KV to 140 KV.

In another embodiment, the step of generating x-ray radiation comprises generating x-rays produced by a source operating at a voltage in the range of 80 KV to 120 KV.

In yet another embodiment, the step of filtering the x-ray radiation is performed with a filter comprising of a 12 mm thick aluminum layer and a 1 mm thick tin layer.

In still another embodiment, the step of filtering the x-ray radiation is performed with a filter comprising a 10 mm thick aluminum layer and a 2.5 mm thick copper layer.

In yet a further embodiment, the step of generating an image comprises generating topogram views.

In a further embodiment, the step of generating an image comprises generating a CT scan image.

In an additional embodiment, the step of generating an image comprises generating two images using two different voltages in the range of 70 KV to 140 KV.

In one more embodiment, the step of generating an image comprises generating a tomographic image.

In a further embodiment, the step of generating an image comprises generating a cone beam image.

In still a further embodiment, the effective dose of the material comprising gadoxeate disodium is a dose of half that of conventional MRI contrast agents used for an abdominal MRI.

According to another aspect, the invention features a method of diagnosing a medical condition of a living subject. The method comprises the steps of: injecting into a blood vessel of a living subject suspected to be experiencing acute cholecystitis an effective dose of a material comprising a hepatobiliary MRI contrast agent that is excreted through the biliary tree into a blood vessel of a living subject suspected to be experiencing acute cholecystitis; selecting suitable x-ray exposure parameters including at least one of beam collimation, x-ray tube current, x-ray tube voltage exposure time, and x-ray beam filtration; generating x-ray radiation; filtering the x-ray radiation to produce filtered x-ray radiation substantially lacking in radiation corresponding to an energy below a characteristic k-absorbtion edge of a heavy atom constituent of the hepatobiliary MRI contrast agent; subjecting the subject to the filtered x-ray radiation; generating an image of the biliary tree and related anatomy of the living subject; determining whether a blockage of the cystic duct is present from the image; and making a diagnosis of the condition of the living subject based on the determination.

In yet a further embodiment, the step of generating an image comprises generating topogram views.

In a further embodiment, the step of generating an image comprises generating a CT scan image.

In an additional embodiment, the step of generating an image comprises generating two images using two different voltages in the range of 70 KV to 140 KV.

In one more embodiment, the step of generating an image comprises generating a tomographic image.

In a further embodiment, the step of generating an image comprises generating a cone beam image.

The foregoing and other objects, aspects, features, and advantages of the invention will become more apparent from the following description and from the claims.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views. All reference to beryllium filtration refers to the beryllium window used in some x-ray tubes. Some computational models of x-ray spectra include beryllium filtration because this option facilitates computation of x-ray spectra at low kV typically for mammography. In this case the inclusion or exclusion of the beryllium does not substantially affect the simulate x-ray spectra and it does not change substantially the effectiveness of the described approach. The vertical line in each spectrum was drawn to indicate the energy at 50.2 keV, which corresponds to the characteristic k-absorbtion edge of gadolinium.

FIG. 1 is a simulated CT x-ray spectrum taken with a tube voltage of 120 kV, with 0.8 mm beryllium and 12 mm aluminum filters.

FIG. 2 is a simulated CT x-ray spectrum taken with a tube voltage of 100 kV, with 0.8 mm beryllium and 12 mm aluminum filters.

FIG. 3 is a simulated CT x-ray spectrum taken with a tube voltage of 80 kV, with 0.8 mm beryllium and 12 mm aluminum filters.

FIG. 4 is a simulated CT x-ray spectrum taken with a tube voltage of 120 kV, with 0.8 mm beryllium, 12 mm aluminum and 1 mm tin filters.

FIG. 5 is a simulated CT x-ray spectrum taken with a tube voltage of 100 kV, with 0.8 mm beryllium, 12 mm aluminum and 1 mm tin filters.

FIG. 6 is a simulated CT x-ray spectrum taken with a tube voltage of 100 kV, with 10 mm aluminum, and 2.5 mm copper filters.

FIG. 7A is an enhanced CT scan image of a liver of a human patient using gadoxetate disodium as a contract agent according to principles of the invention.

FIG. 7B is a gadoxetate disodium-enhanced MRI image of the same patient.

### DETAILED DESCRIPTION

A new medical use of the MRI contrast agent gadoxetate disodium is described, and is believed to be useful to evaluate the patency of the cystic duct by identifying excreted gadoxetate disodium within the gallbladder lumen on regular contrast-enhanced computed tomography (CT) scan obtained to arrive at a diagnosis and “work-up” abdominal pain. We describe the use of gadoxetate disodium as an x-ray intravenously injected contrast medium for imaging the biliary tree and related anatomy using computed tomography (CT) scan. To the best of our knowledge, the use of gadoxetate disodium-enhanced CT scan has never been reported for this purpose.

Gadoxetate disodium (Eovist®, Bayer HealthCare Pharmaceuticals, Wayne, N.J., USA), a relatively new (MRI) contrast agent, was approved by the US Food and Drug Administration for the detection and characterization of focal liver lesions, has gained popularity, due to the relative rapidity in which a hepatobiliary phase can be acquired. With the increasing use of gadoxetate disodium, a wide range of off label clinical applications emerged, focused on the evaluation of the biliary tree using gadoxetate disodium-enhanced MRI. Two recent studies have described the potential use of gadoxetate disodium enhanced MRI to evaluate the cystic duct patency and described the pattern of gallbladder opacification by this particular hepatobiliary contrast agent. Gadoxetate disodium is distributed by Bayer Healthcare under the name “Eovist” in the United States and as “Primovist” in Europe.

Gadolinium-based contrast agents (GBCA) are widely used to enhance tissue contrast in MRI. In the presence of the magnetic field this contrast agent (gadoxetate disodium in this case) enhances the relaxation rate of hydrogen atoms in its vicinity. This is manifested by the shortening of the longitudinal (T1) and transverse (T2) relaxation times but the major increase in the MRI signal is generated by T1 weighting of the image acquisition.

MRI contrast agents were not specifically designed to absorb x-rays. They contain gadolinium as the paramagnetic agent that enhances the MRI signal, and this element also happens to exhibit strong x-ray absorption. Because of their x-ray absorption properties, gadolinium-based agents have been proposed as an alternative to iodinated contrast agents for x-ray planar angiographic and computed tomography (CT) imaging in patients who may not tolerate iodine. However, this substitution is generally not considered prudent in standard practice. See Thomsen H S, Almen T, Morcos S K. Gadolinium-containing contrast media for radiographic examinations: a position paper. Eur Radiol 2002; 12:2600-2605. The following is a direct quotation from the ACR Manual on Contrast Media-Version 9, page 78, American College of Radiology, 2013:
Gadolinium agents are radiodense and can be used for opacification in CT and angiographic examinations instead of iodinated radiographic contrast media. However, there is controversy about whether gadolinium contrast media are less nephrotoxic at equally attenuating doses. Caution should be used in extrapolating the lack of nephrotoxicity of intravenous (IV) gadolinium at MR dosages to its use for angiographic procedures, including direct injection into the renal arteries. No assessment of gadolinium versus iodinated contrast nephrotoxicity by randomized studies of equally attenuating doses is currently available. Initially, radiographic use of high doses of gadolinium agents was proposed as an alternative to nephrotoxic iodinated contrast media in patients with renal insufficiency. However, because of the risk of NSF following gadolinium-based contrast material administration, especially in patients with acute renal failure or severe chronic kidney disease, and because of the unknown nephrotoxicity of high doses of gadolinium agents, use of these contrast media for conventional angiography is no longer recommended.

Currently all gadolinium-based agents are associated with a very small risk of developing nephrogenic systemic fibrosis (NSF) but this is very rare at the lower end of the typical administered dose and it probably does not occur in patients with normal renal function. Although at MRI injected dose levels, gadolinium-based agents are considered extremely safe, exceeding the MRI contrast injected dose of gadolinium agent which is typically needed to perform x-ray imaging is not recommended. According to Bayer Health Care, a dose of 0.1 mL/kg body weight, or 0.025 mmol/kg body weight, is recommended as the standard dosage for MRI. For example, the dose for a 70 kg patient will be 7 ml (1.75 mmol). If a patient is sensitive to iodine and gadolinium is substituted to avoid potential effects from iodine, the high dose of gadolinium required may raise the risk of nephrogenic systemic fibrosis, a very serious debilitating condition. Therefore, one risk is traded for an equal or greater risk and generally this practice of substituting gadolinium for iodine is not recommended.

In most MRI applications the required intravenously injected dose of gadolinium-based agent is much lower than the dose required for x-ray imaging to produce acceptable image contrast. This occurs because gadolinium-based contrast agents contain one atom of gadolinium per molecule compared to iodinated contrast media that contain three iodine atoms per molecule. Therefore, for the typical x-ray imaging application, the injected dose of the gadolinium-based agent must be greatly increased (typically 1.5 to 2 times or more depending on the application) in order to exhibit adequate x-ray absorption and acceptable image quality.

Using iodinated intravenous contrast agent, CT scan fails to image the biliary tree and in particular the gallbladder. Typically, ultrasound and perhaps nuclear medicine cholescintigraphy may follow at least for a fraction of these cases, and these procedures are time consuming and very costly. Nuclear medicine cholescintigraphy is considered the imaging modality of choice for the evaluation of acute cholecystitis based on evaluating the blockage or patency of the cystic duct. Once contrast is seen inside the gallbladder, the diagnosis of acute cholecystitis is excluded, and if contrast fails to get inside the gallbladder, the cystic duct is considered blocked and the patient is diagnosed having acute cholecystitis. Gadoxetate disodium is an MRI contrast agent that is used to assess the functional status of the liver, typically for the diagnosis and work up of hepatic tumors.

Although Gadoxetate disodium-enhanced MRI allows imaging of the biliary tree, MRI is not the appropriate test for work up of abdominal pain and is not available in most emergency departments. Therefore, visualization of excreted biliary contrast (gadoxetate disodium) within the gallbladder on CT scan, which is the modality of choice to work-up patients with abdominal pain, is a significant improvement over current techniques. The novelty of our approach is to be able to use a CT scan to work-up of abdominal pain, and when indicated add to the scanning protocol gadoxetate disodium as a second intravenous contrast to evaluate the cystic duct patency and therefore exclude the possibility of, or diagnose, acute cholecystitis. One important advantage of CT is its high spatial and temporal resolution which enable it to generate images with very high detail compared to ultrasound, nuclear medicine imaging and MRI. There is also the possibility to replace the traditional nuclear medicine cholescintigraphy procedure for many of these cases and image the patient with CT scan but instead of using iodinated contrast we propose to use intravenous injection of gadoxetate disodium for visualization of the biliary tree and related anatomy such as the presence of contrast inside the gallbladder and thus evaluate the patency of the cystic duct.

We believe that we are the first to observe that the gadolinium agent enhances the biliary tree and in particular the gallbladder when used in CT imaging and at lower than body MRI injected contrast dose. This method leads to the diagnosis of acute cholecystitis. Our finding contradicts the dictum that for x-ray imaging, namely that the required injected dose of gadolinium agents is higher than the “safe” dose injected for MRI. We have determined that an effective dose of gadolinium agent is actually about half of that required for an abdominal MRI and this may be further reduced with additional experience. Our finding goes completely against conventional practice, and it is counterintuitive according to the medical literature. Nevertheless, we have data that clearly demonstrates gallbladder opacification following intravenous injection of gadoxetate disodium , under CT imaging conditions.

We disclose a new use of a known contrast agent that can be used under different than usual conditions, and a dosing range that is far different than what would be expected. These observations allow us to perform imaging at levels far below those that are associated with toxicity. This approach is readily translatable to important clinical applications because gadoxetate disodium (Eovist®) is an FDA approved drug but not for CT imaging. In effect, we are proposing the repositioning of an existing drug for a new use under conditions and dose that are drastically different from its original design and intent.

The net benefit is believed to be an improvement in the accuracy of the diagnosis at a greatly reduced time, fast throughput in emergency care, and for a substantial fraction of cases, the ultrasound or nuclear medicine scans (time consuming and costly) will be obviated.

Moreover, imaging of the biliary tree and related anatomy with gadoxetate disodium enhanced CT can be performed under conventional CT imaging parameters at a conventional or at reduced CT radiation dose to the patient. The radiation dose can be reduced by taking advantage of the absorption characteristic k-edge of gadolinium which is at 50.2 keV compared to 33.2 keV for iodine. The x-ray spectrum of the CT x-ray beam can be adjusted for more efficient absorption of x-rays that are closer to the k-edge of gadolinium. This can be accomplished by using a lower tube potential, from the typical of 120 kV to 100kV or even at 70 kV to 80 kV for smaller patients. Moreover, the use of a k-edge filter such as metallic tin or an alloy or compound of tin provides excellent suppression of the x-ray spectrum at energies below 50.2 keV that are not optimal for imaging gadolinium contrast. The combination of lower than standard kV with added k-edge filtration of the x-ray beam will contribute to a substantial radiation dose reduction. It is possible to acquire CT images for this test at greatly reduced radiation and injected dose at approximately (30% or lower radiation and administered dose) than standard levels.

### Image Contrast Optimization And Radiation Dose Reduction

Modern CT scanners can typically operate at x-ray tube potentials from 70 to 150 kilovolts (kV) and at an x-ray tube current from about 10 to 1,300 milliamps (mA). Techniques for using lower voltages (70 kV or 100 kV for example) for increase of image contrast have been described in the literature. However, with a few exceptions, notably in imaging of small children or for brain perfusion CT imaging where 80 kV may be used, 120 kV is the standard for most CT imaging of adult patients.

In the described method using gadoxetate disodium as a CT contrast agent, the usual technique with CT at 120 kV will generate images of acceptable diagnostic quality and radiation dose. In our approach we demonstrate the use of 100 kV, 70 kV and 80 kV for improvement in image contrast, and this technique is expected to be especially beneficial for patients of average to lower than average body habitus. The use of 120 kV is more applicable to patients of above average body habitus. Regardless of the x-ray tube voltage used, image quality and radiation dose with injected gadoxetate disodium in CT can improve substantially by applying additional filtration to the x-ray beam in order to suppress x-rays with energies below the characteristic K-shell absorption of gadolinium which is at 50.2 kilo electron volts (keV). This can be accomplished by adding a combination of aluminum and copper filtration, typically 12 mm and about 2.5 mm respectively. Alternatively, a thin layer of elemental tin or a tin compound in addition to the existing aluminum filters in the x-ray beam. Tin has a characteristic absorption to for x-rays at 29.2 keV and therefore it exhibits strong absorption of x-rays from about 29 to 50 keV. This type of filtration suppresses the x-ray fluence below the characteristic x-ray absorption of gadolinium (50.2 keV). This approach increases the sensitivity of the beam to gadolinium resulting in increased image contrast at a reduced radiation dose. Good results can also be attained by using a combination of copper and aluminum filtration or a combination of aluminum, copper and tin filtration.

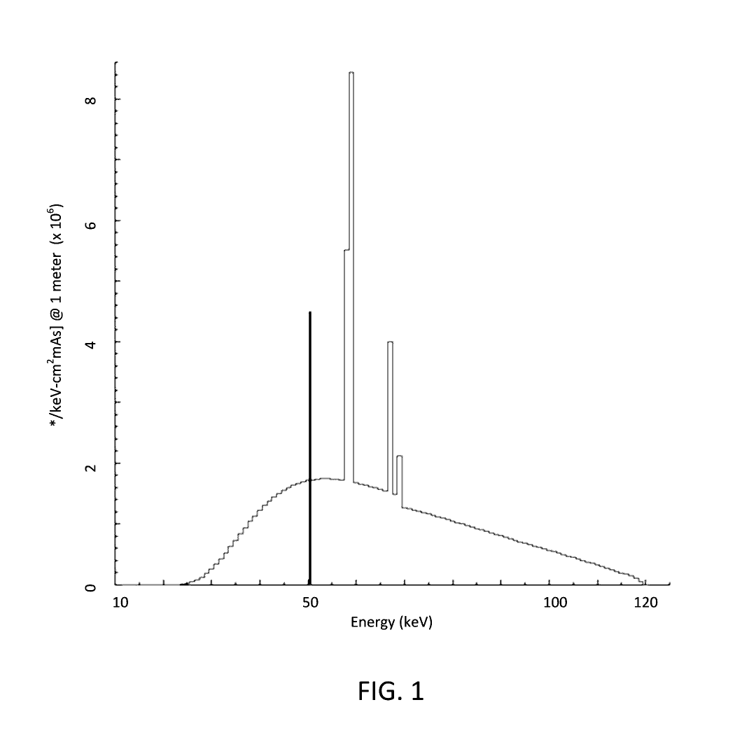

FIG. 1 is a simulated CT x-ray spectrum taken with a tube voltage of 120 kV, with 0.8 mm beryllium and 12 mm aluminum filters. This is a typical spectrum from CT scanners. The vertical line at 50.2 keV points to the energy that corresponds to the characteristic x-ray absorption K-edge of gadolinium. X-rays with energies below 50.2 keV as shown by the vertical line are not optimal for visualizing the injected contrast and they preferably should be suppressed because they contribute to the radiation dose but not substantially to the visualization of the gadolinium agent.

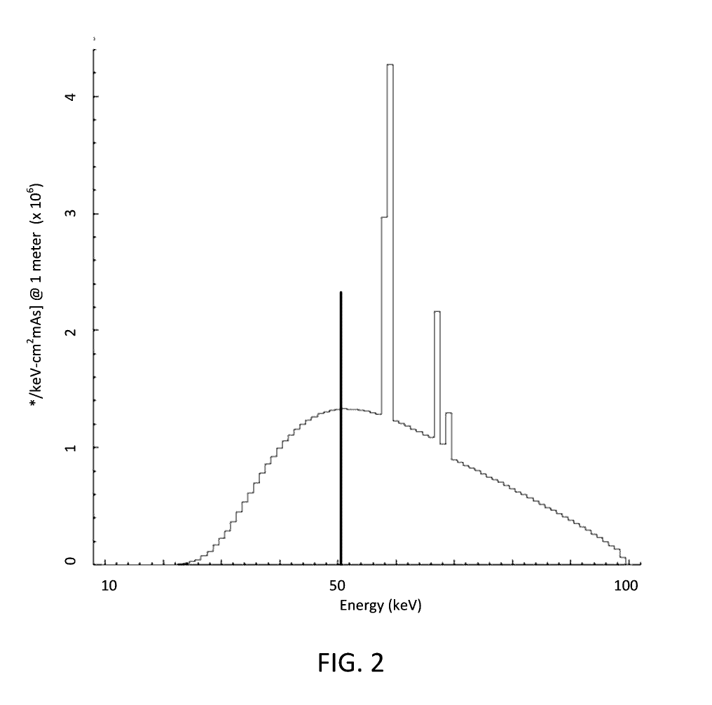

FIG. 2 is a simulated CT x-ray spectrum taken with a tube voltage of 100 kV, with 0.8 mm beryllium and 12 mm aluminum filters.

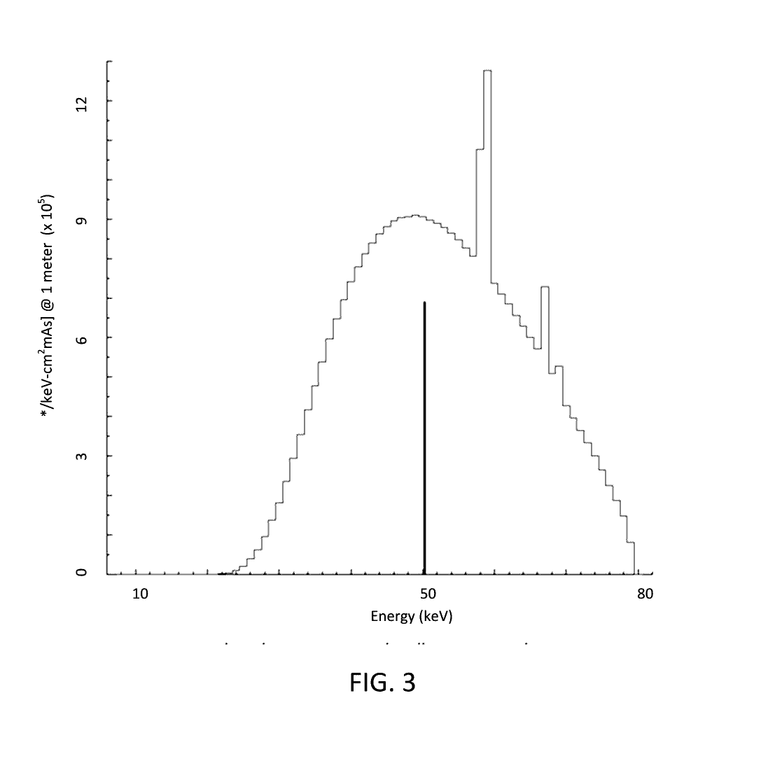

FIG. 3 is a simulated CT x-ray spectrum taken with a tube voltage of 80 kV, with 0.8 mm beryllium and 12 mm aluminum filters.

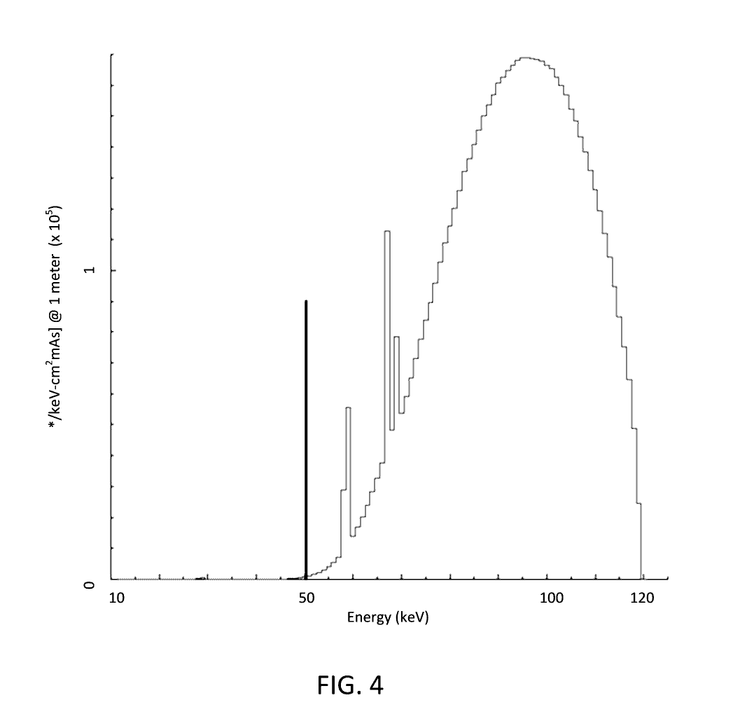

FIG. 4 is a simulated CT x-ray spectrum taken with a tube voltage of 120 kV, with 0.8 mm beryllium, 12 mm aluminum and 1 mm tin filters. In this case, with below 50.2 keV have been filtered out.

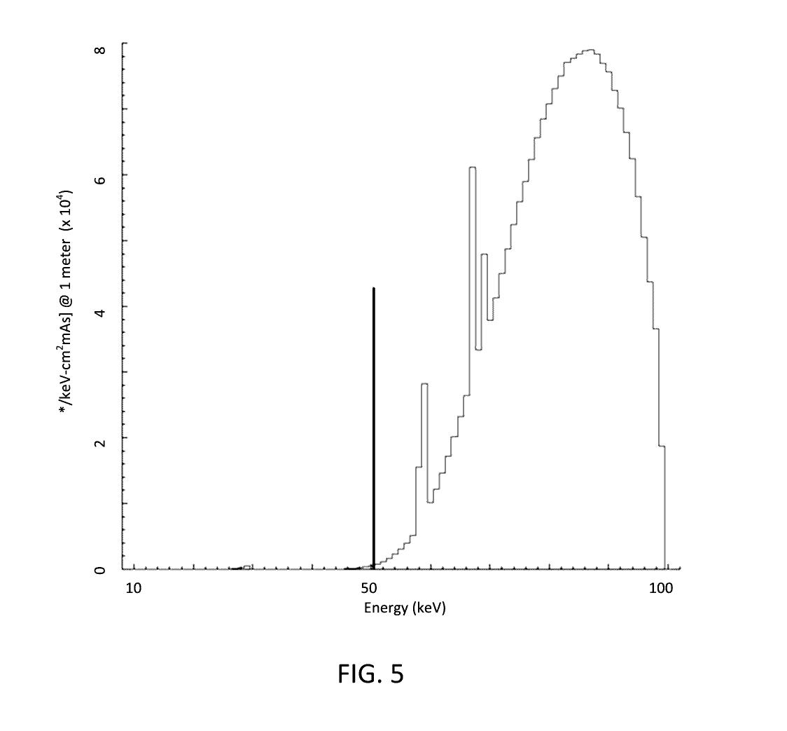

FIG. 5 is a simulated CT x-ray spectrum taken with a tube voltage of 100 kV, with 0.8 mm beryllium, 12 mm aluminum and 1 mm tin filters. In this case, energies below 50.2 keV have been filtered out.

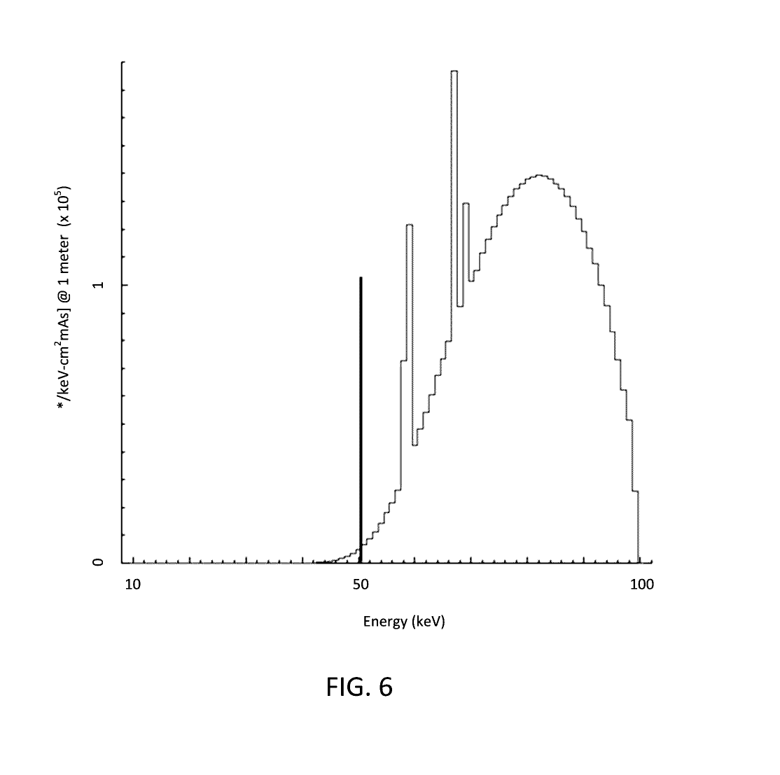

FIG. 6 is a simulated CT x-ray spectrum taken with a tube voltage of 100 kV, with 10 mm aluminum and 2.5 mm copper filters. In this case, energies below 50.2 keV have been filtered out.

In some embodiments, a beryllium window or filter can be omitted because it is only useful for x-ray imaging of relatively small parts of the body such as the breast and it is not needed for CT imaging and for other x-ray imaging studies.

The spectra illustrated in FIG. 1 through FIG. 6 were computed using the SpekCalc simulation program which was developed by Poludniowski et al. The details of this simulation approach have been published in the following references:

Poludniowski GG, Evans P M. Med Phys. 2007 34(6):2164-74.

Poludniowski GG, Med Phys. 2007 34(6):2175-86.

Poludniowski GG, Landry G, DeBlois F, Evans P M, Verhaegen F. Phys Med Biol. 2009 54(19):433-38.

The simulated x-ray spectra illustrated in FIG. 1 through FIG. 6 show how the relative x-ray fluence as a function of energy varies with different peak potential (kV) and x-ray beam filtration. The change of the x-ray spectra with changing kV and filtration is particularly important when examined in reference to the characteristic x-ray absorption (K-shell absorption) which is at 50.2 keV. The preferred spectrum for imaging gadolinium is one that does not contain a high x-ray fluence below 50.2 KeV, the characteristic K-edge absorption of Gd, and it also does not have too many x-rays much above about 100 KeV. An important aspect of enhancing visualization of gadolinium contrast is suppression of the x-ray fluence at energies below the K-absorption edge of gadolinium. FIG. 4, FIG. 5 and FIG. 6 are good examples of the spectra that would contribute to increased contrast and reduction of the radiation dose. It is noted that in FIG. 4, FIG. 5 and FIG. 6 virtually all of the available x-ray energies are above the characteristic absorption K-edge of gadolinium. This is a very desirable condition which enhances efficient absorption of x-rays by the gadolinium-based contrast agent for increased contrast and decreased radiation dose.

The use of lower voltages with or without modification in the x-ray beam filtration reduces the x-ray output of the x-ray tube for a given tube current (measured in milliamps—mA) and exposure time. CT scanners have a wide range of currents and when using lower than 120 kV the current may have to be increased. Alternatively, exposure time per tube rotation may have to be slightly increased if the voltage is too low.

### CT Acquisition Technique

The CT acquisition can be performed at any of the available kilovolt settings of the scanner but the settings from 70 kV to 100 kV are preferred for lower dose. Helical (also called spiral) or axial acquisition can be used with axial or coronal reconstruction and display. Any pitch can be used in the helical mode but generally a higher pitch (generally with a pitch of 1.0 or higher) will be beneficial for dose reduction. A relatively thin x-ray beam collimation of about 5.0 mm is preferred for good x-ray scatter reduction and good contrast but a thicker slice can be used particularly if a fast scan is preferred. Reconstruction can be performed at the highest resolution available but for CT scans that are intended to be a replacement for cholescintigraphy (nuclear medicine test), a lower resolution can be tolerated. The automatic exposure control (also called auto mA) can be used, but caution must be exercised to set the maximum current not at the highest limit to prevent the scanner from delivering higher than desirable dose for this particular scan. Manual exposure control can be used with preset voltage (kV), current (mA), time per rotation and pitch. Dose reduction techniques such as model based image reconstruction or partial scanning (less than 360 degree acquisition) can be used.

### Other X-Ray Techniques

Images of the biliary anatomy using a gadolinium agent like the gadoxetate disodium can be also acquired using the following techniques:

Scout (also called topogram) views with CT.

Dual energy CT and spectral decomposition, and spectral photon counting acquisition. The dual-energy technique uses two kV settings for better material and tissue discrimination. The spectral decomposition technique generates a virtual monochromatic spectrum from a conventional x-ray spectrum. Other approaches such as the spectral photon counting technique uses detectors that count individual x-ray events and generate an x-ray spectrum. Single, dual or multiple x-ray energy analysis can be performed for better characterization of contrast material from tissues.

Digital tomosynthesis. This is a particularly promising but never reported technique for biliary imaging using an agent such as gadoxetate disodium. In this approach, typically about 8 to 25 radiographic exposures are acquired and a tomographic image is reconstructed. These images are typically reconstructed as coronal views that are ideally suited for visualizing the biliary anatomy. The main advantage of tomosynthesis over CT is in the lower radiation dose and potentially lower cost.

Cone Beam CT. Cone beam CT is computed tomography using a flat panel detector with a relatively large area rather than a narrow shaped (fan) beam. Cone beam does not produce very good contrast but it can be very convenient at some medical facilities. Interestingly, unlike conventional CT, filters of any kind can be easily added in cone beam CT and in digital tomosynthesis systems.

A second clinical use of gadoxetate disodium, using computed tomography which is applicable to a much larger population of patients compared to its current use with MRI. Given the large number of cases seen in the average healthcare facility with upper quadrant abdominal pain and suspected cholecystitis, typically in the Emergency Department, the proposed approach solves a very important problem and it greatly shortens the duration of the diagnostic process. Considering the fact that gadoxetate disodium is already FDA approved and it is considered among the safest of GBCAs its commercial potential for the new use we describe is very high.

### EXAMPLE

A 51-year-old female patient was undergoing evaluation for living liver donation.

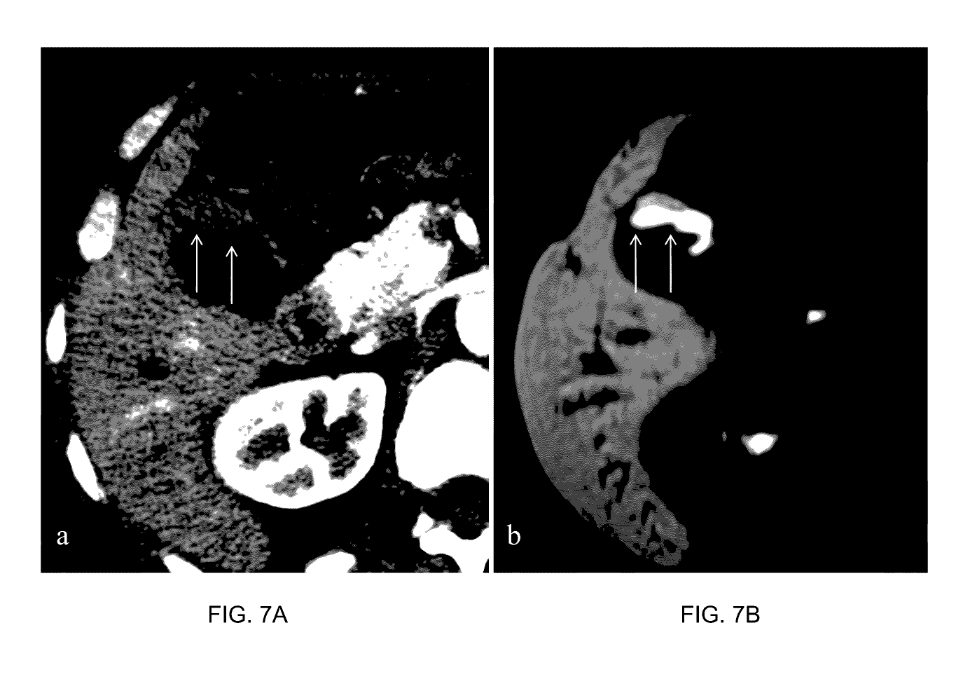

FIG. 7A is an enhanced CT scan image of her liver using gadoxetate disodium as a contract agent according to principles of the invention. FIG. 7A, obtained during the arterial phase for the evaluation of the vascular anatomy, demonstrates excreted hepatobiliary contrast (gadoxetate disodium) as an anti-dependant hyperdensity within the gallbladder fundus (white arrows). In this particular patient the CT scan was obtained 99 minutes after the gadoxetate disodium-enhanced MRI shown in FIG. 7B, a relatively long interval to image gallbladder filling. A better contrast resolution is expected when using shorter interval. The highest concentration of contrast within the gallbladder, and therefore the best contrast resolution, is expected between 30 to 60 minutes from the time of intravenous injection of contrast.

The CT scan technique used included a slice thickness of 4.00 mm, and a field of view of 283.0 mm. The x-ray radiation was generated using 120 kV and a current of 182 mA, with a tube current of 182 mA and tube current-time product of 213 mAs.

FIG. 7B is a gadoxetate disodium-enhanced MRI image of the same patient. FIG. 7B demonstrates the excreted contrast within the gallbladder lumen (white arrows) which correlates with the anti-dependant hyperdense contrast seen on the CT scan of FIG. 7A. The MRI of FIG. 7B was obtained 20 minutes following intravenous administration of gadoxetate disodium.

After injecting the living subject, the x-ray exposure using CT is initiated by selecting the proper acquisition mode. In the simplest case, this requires selection of the scanning mode (spiral or axial), the field of view, x-ray collimation, x-ray tube voltage (kV), the x-ray tube current in milliamps (mA), the speed of rotation, the beam pitch, and the section thickness.

There is also the automatic exposure mode (which is used most of the time in modern CT scanners) that dynamically modulates the x-ray tube current (mA) during the scan for optimal exposure and radiation dose reduction. Current CT scanners do not allow a change in the x-ray beam filtration by the operator although the filtration can change automatically when the operator changes the field of view (head versus body) for example. Some CT scanners may change the filtration automatically if the operator selects a particular kV setting, changing to 100 kV from 120 kV, for example. Changing the type of filter by the operator is not practiced today and it is very unlikely in the future.

In some embodiments, a tube voltage of from 100 kV to 120 kV with the body field of view and filtration that is provided in the scanner (typically between 7 to 10 mm of aluminum) may be used to make CT scans according to the principles of the invention.

### Definitions

Unless otherwise explicitly recited herein, any reference to an electronic signal or an electromagnetic signal (or their equivalents) is to be understood as referring to a non-volatile electronic signal or a non-volatile electromagnetic signal.

Unless otherwise explicitly recited herein, any reference to “record” or “recording” is understood to refer to a non-volatile or non-transitory record or a non-volatile or non-transitory recording.

### Theoretical Discussion

Although the theoretical description given herein is thought to be correct, the operation of the devices described and claimed herein does not depend upon the accuracy or validity of the theoretical description. That is, later theoretical developments that may explain the observed results on a basis different from the theory presented herein will not detract from the inventions described herein.

Any patent, patent application, patent application publication, journal article, book, published paper, or other publicly available material identified in the specification is hereby incorporated by reference herein in its entirety. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

While the present invention has been particularly shown and described with reference to the preferred mode as illustrated in the drawing, it will be understood by one skilled in the art that various changes in detail may be affected therein without departing from the spirit and scope of the invention as defined by the claims.

What is claimed is:

1. A method of diagnosing a medical condition of a living subject, comprising the steps of:
injecting into a blood vessel of a living subject suspected to be experiencing acute cholecystitis an effective dose of a material comprising gadoxetate disodium;
selecting suitable x-ray exposure parameters including at least one of beam collimation, x-ray tube current, x-ray tube voltage exposure time, and x-ray beam filtration;
generating x-ray radiation;
filtering said x-ray radiation to produce filtered x-ray radiation substantially lacking in radiation corresponding to an energy below 50.2 KeV;
subjecting said subject to said filtered x-ray radiation;
generating an image of the biliary tree and related anatomy of said living subject;
determining whether a blockage of the cystic duct is present from said image; and
making a diagnosis of the condition of said living subject based on said determination.

2. The method of diagnosing a medical condition of a living subject of claim 1, further comprising the step of recording said image, transmitting said image to a data handling system, or to displaying said image to a user.

3. The method of diagnosing a medical condition of a living subject of claim 1, wherein said step of generating x-ray radiation comprises generating x-rays produced by a source operating at a voltage in the range of 70 KV to 140 KV.

4. The method of diagnosing a medical condition of a living subject of claim 1, wherein said step of generating x-ray radiation comprises generating x-rays produced by a source operating at a voltage in the range of 80 KV to 120 KV.

5. The method of diagnosing a medical condition of a living subject of claim 1, wherein said step of filtering said x-ray radiation is performed with a filter comprising a 12 mm thick aluminum layer and a 1 mm thick tin layer.

6. The method of diagnosing a medical condition of a living subject of claim 1, wherein said step of filtering said x-ray radiation is performed with a filter comprising a 10 mm thick aluminum layer and a 2.5 mm thick copper layer.

7. The method of diagnosing a medical condition of a living subject of claim 1, wherein said step of generating an image comprises generating topogram views.

8. The method of diagnosing a medical condition of a living subject of claim 7, wherein said step of generating an image comprises generating a CT scan image.

9. The method of diagnosing a medical condition of a living subject of claim 1, wherein said step of generating an image comprises generating two images using two different voltages in the range of 70 KV to 140 KV.

10. The method of diagnosing a medical condition of a living subject of claim 1, wherein said step of generating an image comprises generating a tomographic image.

11. The method of diagnosing a medical condition of a living subject of claim 1, wherein said step of generating an image comprises generating a cone beam computed tomography image.

12. The method of diagnosing a medical condition of a living subject of claim 1, wherein said effective dose of said material comprising gadoxeate disodium is a dose of half that used for an abdominal MRI using conventional MRI contrast agents.

13. A method of diagnosing a medical condition of a living subject, comprising the steps of:
injecting into a blood vessel of a living subject suspected to be experiencing acute cholecystitis an effective dose of a material comprising a hepatobiliary MRI contrast agent that is excreted through the biliary tree;
selecting suitable x-ray exposure parameters including at least one of beam collimation, x-ray tube current, x-ray tube voltage, exposure time, and x-ray beam filtration;
generating x-ray radiation;
filtering said x-ray radiation to produce filtered x-ray radiation substantially lacking in radiation corresponding to an energy below a characteristic k-absorbtion edge of a heavy atom constituent of said hepatobiliary MRI contrast agent;
subjecting said subject to said filtered x-ray radiation;
generating an image of the biliary tree and related anatomy of said living subject;
determining whether a blockage of the cystic duct is present from said image; and
making a diagnosis of the condition of said living subject based on said determination.

14. The method of diagnosing a medical condition of a living subject of claim 13, wherein said step of generating an image comprises generating topogram views.

15. The method of diagnosing a medical condition of a living subject of claim 14, wherein said step of generating an image comprises generating a CT scan image.

16. The method of diagnosing a medical condition of a living subject of claim 13, wherein said step of generating an image comprises generating two images using two different voltages in the range of 70 KV to 140 KV.

17. The method of diagnosing a medical condition of a living subject of claim 13, wherein said step of generating an image comprises generating a tomographic image.

18. The method of diagnosing a medical condition of a living subject of claim 13, wherein said step of generating an image comprises generating a cone beam computed tomography image.
